(19) United States
(12) Patent Application Publication
(10) Pub. No.: US 2026/0272316 A1
(43) Pub. Date: Sep. 17, 2026

(54) METHOD, DEVICE AND RECORDING MEDIUM FOR ESTIMATING FUNCTIONAL THRESHOLD POWER BASED ON MULTIPLE MODELS

(71) Applicant: RIDUCK INC., Seoul (KR)

(72) Inventor: Sanghyuk PARK, Seoul (KR)

(73) Assignee: RIDUCK INC., Seoul (KR)

(21) Appl. No.: 19/667,816

(22) Filed: May 5, 2026

Related U.S. Application Data
(63) Continuation-in-part of application No. PCT/KR2025/009982, filed on Jul. 9, 2025.

(30) Foreign Application Priority Data
Aug. 13, 2024 (KR) 10-2024-0108561
Oct. 24, 2024 (KR) 10-2024-0146234

(51) Int. Cl. A61B 5/024; A61B 5/00

(52) U.S. Cl. CPC A61B 5/024; A61B 5/7267; A61B 2503/10

(57) ABSTRACT

A method of estimating functional threshold power (FTP) on the basis of multiple models is disclosed to achieve the above-described object. The method includes acquiring time-series user data, selecting a single model among a plurality of FTP estimation models as a user-customized FTP estimation model on the basis of the time-series user data, and generating FTP information by utilizing the user-customized FTP estimation model. The time-series user data includes time-power data and heart rate data generated during a user's ride, and the plurality of FTP estimation models are models configured to estimate the FTP information in different ways.

### CROSS REFERENCE TO RELATED APPLICATIONS

The present application is a Continuation-in-Part of International Application No. PCT/KR2025/009982 filed on Jul. 9, 2025, which is based upon and claims the benefit of priority to Korean Patent Application No. 10-2024-0108561 filed on Aug. 13, 2024 and Korean Patent Application No. 10-2024-0146234 filed Oct. 24, 2024, the entire contents of which are incorporated herein by reference.

### BACKGROUND

### 1. Technical Field

The present invention relates to a method of estimating functional threshold power (FTP) on the basis of multiple models, and more particularly, to a method, device, and computer program for selecting an appropriate one of a plurality of FTP estimation models by analyzing a user's time-series data and generating user-customized FTP information by utilizing the selected model.

### 2. Related Art

Functional threshold power (FTP) is the power that may be sustained for 20 to 70 minutes, depending on training level, at the exercise intensity (maximal lactate steady state (MLSS)) where lactate in the body remains stable. This is now widely used as a key indicator for managing bicycle races and training. Since FTP provides essential data for evaluating cycling performance and designing a training plan, it is very important for various training platforms and equipment to include an FTP estimation function. This enables efficient analysis of a user's fitness level and physical condition and the design of a personalized training plan.

The most common of currently used FTP measurement methods is based on an FTP test protocol. The most widely known method is a 20-minute FTP test, where a power value is estimated at exercise intensity at which a lactate level in the body remains stable (a maximal lactate steady state (MLSS)), by multiplying an average power measured during 20 minutes of exerting maximal effort by a correction factor of 0.95. In addition to these, there are methods such as a ramp test where power is gradually increased, a method of measuring a mean maximal power (MMP), and the like. However, these methods may yield varying results depending on a user's physical condition or a test protocol. Particularly for general users, the methods have the disadvantage of being burdensome and difficult to perform.

Lately, a submaximal FT estimation method utilizing the correlation between heart rate and power has emerged. This method has the advantages of enabling estimation based on data collected while a user is riding at moderate intensity or higher, without requiring FTP testing in person. However, heart rate varies significantly among individuals and is easily influenced by environmental factors, imposing limitations on accuracy.

In particular, existing FTP estimation methods rely on a fixed test protocol, often failing to adequately reflect changes in a user's riding pattern, physical condition, training level, test environment, and the like. For example, a user's actual FTP may be overestimated in riding at high intensity for a short duration, while it may be underestimated in riding at low intensity for a long duration. To address these issues, a data analysis method in which various factors such as a heart rate, a peak power, a riding time, and an exercise intensity pattern are comprehensively considered is required.

Methods of estimating FTP by analyzing multiple peak power values or utilizing the correlation between heart rate and power continue to be researched, but these are also limited in that they exhibit significant individual variation and fail to provide consistent results for all users. Therefore, a new approach is required for reflecting a variety of user conditions and a user's riding pattern more precisely.

### SUMMARY

The present invention has been developed in response to the foregoing background technology, and the present invention is directed to providing a method, device, and computer program for estimating personalized functional threshold power (FTP) information more accurately by reflecting a user's riding pattern, physical condition, and training level more precisely.

Objects to be achieved by the present invention are not limited to that described above, and other objects that have not been described will be clearly understood by those of ordinary skill in the art from the following description.

To achieve the above-described purpose, a method of estimating functional threshold power (FTP) on the basis of multiple models is provided according to an embodiment of the present invention. The method includes acquiring time-series user data, selecting a single model among a plurality of FTP estimation models as a user-customized FTP estimation model on the basis of the time-series user data, and generating FTP information by utilizing the user-customized FTP estimation model. The time-series user data includes time-power data and heart rate data generated during a user's ride, and the plurality of FTP estimation models are models configured to estimate the FTP information in different ways.

According to an alternative embodiment, the plurality of FTP estimation models may include a first model configured to estimate FTP on the basis of time-series data corresponding to an FTP test protocol, a second model configured to estimate FTP on the basis of mean maximal power (MMP) data, and a third model configured to estimate FTP on the basis of a correlation between heart rate data and power data.

According to an alternative embodiment, the selecting of the single model among the plurality of FTP estimation models may include detecting behavioral intention on the basis of the time-series user data by utilizing a classification model, when the behavioral intention is detected, selecting one of the first model and the second model as the user-customized FTP estimation model, and when the behavioral intention is not detected, selecting the third model as the user-customized FTP estimation model, and the behavioral intention may include information related to whether maximal effort is intentionally exerted and whether an FTP test is performed.

According to an alternative embodiment, the classification model may be a neural network model that is pretrained using a training dataset including time-series data and riding data of a plurality of users and intention information corresponding thereto, and may detect the behavioral intention by analyzing the time-series user data.

According to an alternative embodiment, the selecting of one of the first model and the second model as the user-customized FTP estimation model when the behavioral intention is detected, may include selecting the first model as the user-customized FTP estimation model when intention information related to performing of the FTP test is detected, and selecting the second model as the user-customized FTP estimation model when intention information related to the intentional maximal effort is detected.

According to an alternative embodiment, the first model may calculate an average power corresponding to a specific riding interval included in the time-series data, identify a type of test in accordance with a riding pattern, identify a correction factor corresponding to the identified type of test, and generate the FTP information on the basis of the calculated average power and the identified correction factor.

According to an alternative embodiment, the second model may analyze the user's weight and riding interval data on the basis of the MMP data to derive correlation information between the MMP and the weight, generate a power duration curve on the basis of the derived correlation information, and generate the FTP information on the basis of the generated power duration curve.

According to an alternative embodiment, the third model may calculate an average power of a stable heart-rate interval by analyzing a correlation between the user's heart rate and power and estimate FTP information on the basis of the calculated average power and the user's maximum heart rate.

According to an alternative embodiment, outputs of the plurality of models may have priorities in an order of the first model, the second model, and the third model, and the method may further include updating the FTP information when FTP information higher than FTP information of a previous time point is generated.

According to an alternative embodiment, the method may further include estimating time to exhaustion (TTE) information on the basis of the FTP information and weight data and applying an anaerobic weight on the basis of user information to correct the TTE information and generate customized TTE information. The customized TTE information may be information defining a time for which a maximum power is maintained by the user.

According to another embodiment of the present invention, a device for performing a multi-model-based FTP estimation method is disclosed. The device includes a memory configured to store one or more instructions and a processor configured to execute the one or more instructions stored in the memory. The processor may execute the one or more instructions to perform the multi-model-based FTP estimation method.

According to another embodiment of the present invention, a computer program recorded on a computer-readable recording medium to perform a multi-model-based FTP estimation method is disclosed. The computer program performs the multi-model-based FTP estimation method in combination with a computer which is hardware.

Other details of the present invention are included in the detailed description and drawings.

### BRIEF DESCRIPTION OF DRAWINGS

Various aspects will now be described with reference to drawings, wherein similar reference numerals are used to indicate similar components throughout. In the following embodiments, for illustrative purposes, numerous specific details are presented to provide a comprehensive understanding of one or more aspects. However, it is obvious that such an aspect may be implemented without these particular details.

FIG. 1 is an exemplary diagram schematically showing a system for implementing a multi-model-based functional threshold power (FTP) estimation method according to an embodiment of the present invention.

FIG. 2 is a hardware configuration diagram of a computing device for performing a multi-model-based FTP estimation method according to an embodiment of the present invention.

FIG. 3 is a flowchart illustrating a multi-model-based FTP estimation method according to an embodiment of the present invention.

FIGS. 4 to 6 are various exemplary views of time-series user data according to an embodiment of the present invention.

FIGS. 7 and 8 are exemplary flowcharts illustrating a process of selecting a user-customized FTP estimation model depending on a behavioral intention detection result according to an embodiment of the present invention.

### DESCRIPTION OF EXAMPLE EMBODIMENTS

The advantages and features of the present invention and a method of achieving them will become apparent from embodiments which will be described in detail below with reference to the accompanying drawings. However, the present invention is not limited to the embodiments disclosed below and may be implemented in various different forms. The embodiments are only provided to make the disclosure of the present invention complete and fully convey the scope of the present invention to those skilled in the technical field to which the present invention pertains. The present invention is only defined by the scope of the claims.

Terminology used in this specification is only for the purpose of describing embodiments and is not intended to limit the present invention. In this specification, singular forms include plural forms as well unless the context particularly indicates otherwise. The terms “comprises” and/or “comprising” used in this specification do not preclude the presence or addition of one or more components other than stated components. Throughout the specification, like reference numerals refer to like components, and the term “and/or” includes any of stated components or any combination thereof. Although the terms “first,” “second,” etc., may be used to describe various components, these components are not limited by the terms. These terms are used for the sole purpose of distinguishing one component from others. Therefore, a first component mentioned below may be a second component within the technical scope of the present invention.

Unless defined otherwise, all terms used herein (including technical or scientific terms) have the same meanings as those generally understood by those skilled in the technical field to which the present invention pertains. In addition, terms defined in commonly used dictionaries are not interpreted ideally or excessively unless clearly and particularly defined herein.

As used herein, the term “unit” or “module” refers to a software component or a hardware component such as a field programmable gate array (FPGA) or an application-specific integrated circuit (ASIC), and a “unit” or “module” performs certain roles. However, a “unit” or “module” is not limited to software or hardware. A “unit” or “module” may be configured to reside in an addressable storage medium or run on one or more processors. Accordingly, as an example, a “unit” or “module” includes components, such as software components, object-oriented software components, class components, and task components, processes, functions, attributes, procedures, subroutines, segments of program code, drivers, firmware, microcode, circuitry, data, databases, data structures, tables, arrays, and variables. Components and functions provided in “units” or “modules” may be combined into a smaller number of components, and “units” or “modules” may be subdivided into additional components and “units” or “modules.”

In this specification, a computer is any type of hardware device including at least one processor and may be understood as collectively including a software configuration operating in the corresponding hardware device according to embodiments. For example, a computer may be understood as, but is not limited to, a meaning including all of a smartphone, a tablet personal computer (PC), a desktop computer, a notebook computer, and a user client and an application running on each device.

Those of ordinary skill in the art should appreciate that various illustrative logical blocks, configurations, modules, circuits, and algorithm steps described in connection with embodiments disclosed herein may be implemented as electronic hardware, computer software, or combinations of both. To clearly illustrate this interchangeability of hardware and software, various illustrative components, blocks, configurations, means, logics, modules, circuits, and steps have been described above generally in terms of their functionality. Whether such functionality is implemented as hardware or software depends on a particular application and design constraints imposed on the overall system. Those skilled in the art may implement the described functionality in various ways for each particular application. However, such implementation decisions should not be interpreted as causing a departure from the scope of the present invention.

Hereinafter, embodiments of the present invention will be described in detail with reference to the accompanying drawings.

Each step described in this specification is described as being performed by a computer, but the subject of each step is not limited thereto. At least some steps may be performed in different devices according to embodiments.

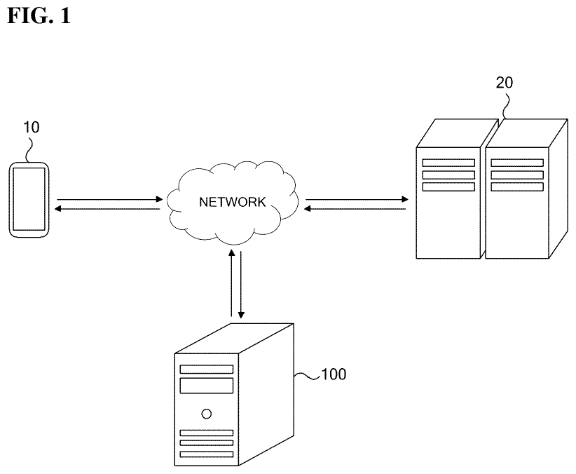

FIG. 1 is an exemplary diagram schematically showing a system for implementing a multi-model-based functional threshold power (FTP) estimation method.

As shown in FIG. 1, a system according to an embodiment of the present invention may include a computing device 100, a user terminal 10, an external server 20, and a network. The components shown in FIG. 1 are illustrative, and additional components may exist, or some of the components shown in FIG. 1 may be omitted. The computing device 100, the external server 20, and the user terminal 10 according to embodiments of the present invention may mutually transmit and receive data for the system according to embodiments of the present invention via the network.

The system according to embodiments of the present invention may include the computing device 100, the user terminal 10, the external server 20, and the network. The computing device 100, the user terminal 10, and the external server 20 according to embodiments of the present invention may mutually transmit and receive data for the system according to embodiments of the present invention.

As the network according to embodiments of the present invention, various wired communication systems such as public switched telephone network (PSTN), an x digital subscriber line (xDSL), a rate adaptive DSL (RADSL), a multi-rate DSL (MDSL), a very high speed DSL (VDSL), a universal asymmetric DSL (UADSL), a high bit rate DSL (HDSL), a local area network (LAN), etc., may be used.

Also, the network presented herein may employ various wireless communication systems such as a code division multiple access (CDMA) system, a time division multiple access (TDMA) system, a frequency division multiple access (FDMA) system, an orthogonal frequency division multiple access (OFDMA) system, a single carrier (SC)-FDMA system, and other systems.

The network according to embodiments of the present invention may be constructed using any communication modality such as wired communication, wireless communication, etc., and may be configured as various communication networks such as a personal area network (PAN), a wide area network (WAN), and the like. Also, the network 400 may be the well-known World Wide Web (WWW) and may employ a wireless transmission technique used for short-range communication such as Infrared Data Association (IrDA) or Bluetooth. Technologies described in this specification may be used not only in the foregoing networks but also in other networks.

According to an embodiment of the present invention, the computing device 100 for performing a multi-model-based FTP estimation method (hereinafter “computing device 100”) may detect whether maximal effort is intentionally exerted, on the basis of time-series user data and select an optimal model among a plurality of FTP estimation models to generate user-customized FTP information.

In the present invention, FTP information may refer to the power of an exercise intensity at which a lactate level in a user's body is stably maintained (a maximal lactate steady state (MLSS)), and this power may be continuously exerted for 20 to 70 minutes depending on an individual. FTP information may be utilized for evaluating cycling performance or designing a training plan and may also be used as an important indicator for evaluating a user's fitness level and analyzing a capability to continuously maintain exercise intensity. In addition, FTP information may be utilized in various sports and fitness-related activities such as setting a training goal and predicting competition performance.

In the present invention, time-series user data relates to time-power data, heart rate data, and changes in exercise intensity occurring during the user's ride. This may include, for example, power output and heart rate changes during a 20-minute FTP test, or data from daily rides. As an example, the user's ride refers to exercise activities recorded during outdoor cycling or indoor trainer use, including power output, heart rate changes, riding speed, and distance recorded in real time in accordance with exercise intensity. Such time-series data is collected in real time by external devices (e.g., a power meter, a heart rate meter, a speedometer, a Global Positioning System (GPS) device, etc.), and the data is transmitted to the computing device 100 and may be used for analyzing the user's riding pattern, selecting an appropriate FTP estimation model, and generating user-customized FTP information.

Specifically, the computing device 100 may collect time-power data, heart rate data, etc., generated during the user's ride in real time, detect whether maximal effort is intentionally exerted on the basis of the collected data, and select an optimal model among a plurality of FTP estimation models in accordance with the detection result, generating user-customized FTP information.

More specifically, the computing device 100 selects one of a plurality of FTP estimation models for generating FTP information in different ways as a user-customized FTP estimation model and generates optimal FTP information on the basis of the user's time-series data via the user-customized FTP estimation model. According to an embodiment of the present invention, the plurality of FTP estimation models may include a first model complying with FTP test protocol, a second model utilizing mean maximal power (MMP) data, and a third model utilizing the correlation between heart rate and power.

The computing device 100 analyzes the user's riding pattern, time-power data, and heart rate data to select the optimal model among the plurality of FTP estimation models. The computing device 100 utilizes a classification model to detect, in the time-series user data, whether maximal effort is intentionally exerted or whether an FTP test is performed and selects the most appropriate FTP estimation model on the basis of the detection result.

Here, the classification model is a pretrained neural network model that determines which model to use when a specific pattern is detected during a ride in data of a plurality of users and trained patterns. For example, when an intentional FTP test is detected, the first model may be selected, when MMP is detected, the second model may be selected, and when the riding pattern is general, the third model may be selected to generate user-customized FTP estimation information.

In other words, the computing device 100 can provide more accurate and personalized FTP information by selecting one of the plurality of FTP estimation models as a user-customized FTP estimation model.

According to an embodiment, to address the data bias issues, the present invention implements multi-model-based FTP estimation that reflects riding data and individual characteristics of various users. Existing FTP estimation methods are based on data from trained elite athletes or performed in accordance with fixed testing protocols, thus often yielding results unsuitable for general users. However, according to the present invention, personalized FTP estimation is performed to reflect the user's actual riding data, heart rate data, and various variables such as weight, age, and gender.

In particular, by dynamically selecting an appropriate model for each user among a plurality of FTP estimation models using a classification model, it is possible to move beyond the existing uniform data-based estimation approach and estimate FTP in individualized ways for all users. For example, for general users, a method of estimating FTP on the basis of the correlation between heart rate and power during daily riding patterns may be utilized, while for trained athletes, a method based on high-intensity test data may be applied.

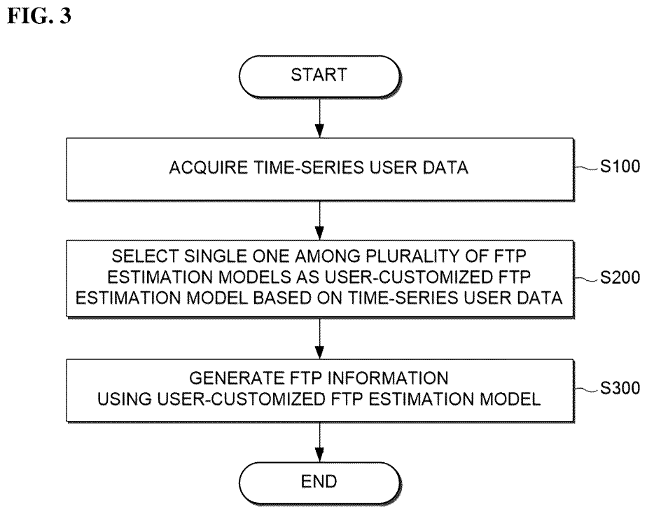

This eliminates data bias corresponding only to specific user groups (e.g., trained user groups), enabling general users to obtain accurate FTP information through individually optimized models. In other words, customized FTP estimation is provided to users at different levels such that a universal system applicable to all users without any data bias can be implemented. Further, a method provided by the computing device 100 of the present invention enables natural FTP information estimation based on daily riding data and reduces dependency on an existing fixed test protocol. The multi-model-based FTP estimation method of the present invention and its effects will be described in detail below with reference to FIG. 3.

According to an embodiment, only the single computing device 100 is shown in FIG. 1, but it is apparent to those of ordinary skill in the art that more servers may be included within the scope of the present invention and the computing device 100 may include additional components. That is, the computing device 100 may be composed of a plurality of computing devices. In other words, a set of a plurality of nodes may constitute the computing device 100.

According to an embodiment of the present invention, the computing device 100 may be a server that provides a cloud computing service. More specifically, the computing device 100 may be a server that provides, as a type of Internet-based computing, a cloud computing service of processing information using another computer connected to the Internet rather than the user's computer. The cloud computing service may be a service for storing data on the Internet and making data or programs available to a user anytime and anywhere through Internet access without requiring the user to install the necessary data or programs on his or her own computer, and may facilitate sharing and transmitting data stored on the Internet with simple operations and clicks. Also, the cloud computing service may be a service that is not only for storing data on a server on the Internet but also for performing desired tasks using the functions of application programs provided on the web without installing programs and enables several people to simultaneously share documents and work together. In addition, the cloud computing service may be implemented in the form of at least one of an infrastructure as a service (IaaS), a platform as a service (PaaS), software as a service (SaaS), a virtual machine-based cloud server, and a container-based cloud server. In other words, the computing device 100 of the present invention may be implemented in the form of at least one of the foregoing cloud computing services. The foregoing specific cloud computing services are merely illustrative, and any platform for constructing a cloud computing environment of the present invention may be included.

According to an embodiment of the present invention, the user terminal 10 is a device that may receive a user's FTP estimation information and exercise achievement analysis information by exchanging information with the computing device 100, and may be a mobile device held by the user. For example, the user terminal 10 may be a device related to a user who tries to acquire exercise achievement and FTP information. Also, the user terminal 10 is a device that may interoperate with an indoor trainer or bicycle to collect riding data and heart rate data in real time, and may serve to provide exercise-related information to the user.

The user terminal 10 helps the design of training plans and the evaluation of riding performance by providing FTP information and exercise achievement analysis information acquired from the computing device 100 to the user. For example, the user terminal 10 may provide information on peak power and an FTP value of each riding interval to the user and may also be utilized as a device for providing personalized feedback in accordance with training performance.

Also, the user terminal 10 may be connected to the computing device 100 or the external server 20 to transmit exercise data in real time or receive comprehensive analysis information of exercise results. For example, the user terminal 100 may transmit data such as heart rate changes, time-power data, and exercise intensity to the external server 20 and receive results based on the user's exercise capabilities.

The external server 20 may be a server for collecting the user's riding data and exercise data and providing a comprehensive analysis, and may provide the user with exercise achievement evaluation, FTP estimation, and time to exhaustion (TTE) information. For example, the external server 20 calculates a more accurate correction factor by comparatively analyzing data of various users and supports exercise planning and performance evaluation on the basis of the correction factor.

In FIG. 1, the external server 20 and the computing device 100 are shown as separate entities. However, according to an embodiment of the present invention, the computing device 100 may be incorporated into the external server 20, and the single device may perform FTP estimation, exercise achievement analysis, and result provision functions. In this case, the user terminal 10 may access the external server 20 to analyze the user's exercise achievement and design a personalized exercise plan.

In addition, when the computing device 100 and the external server 20 are separated, the computing device 100 may provide the user's exercise capability information to the external server 20, and the external server 20 may generate analysis results in accordance with the user's exercise achievement on the basis of the user's exercise capability information. In this way, the user terminal 10 may receive comprehensive feedback on actual riding performance in connection with the user's actual exercise data.

The external server 20 is a digital device that includes a processor and memory for computation capability such as a laptop computer, a notebook computer, a desktop computer, a web pad, and a mobile phone. The external server 20 may be a web server for processing service. The foregoing types of servers are merely illustrative, and the present invention is not limited thereto.

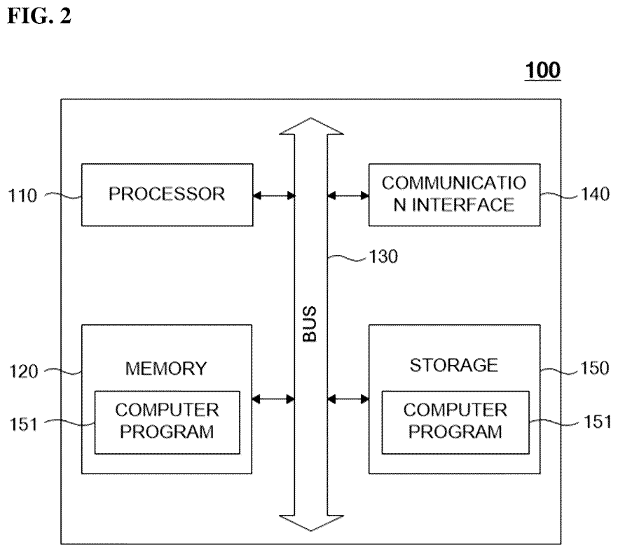

FIG. 2 is a hardware configuration diagram of a computing device for performing the multi-model-based FTP estimation method according to an embodiment of the present invention.

Referring to FIG. 2, the computing device 100 for performing the multi-model-based FTP estimation method according to the embodiment of the present invention may include at least one processor 110, a memory 120 into which a computer program 151 executed by the processor 110 is loaded, a bus 130, a communication interface 140, and a storage 150 in which the computer program 151 is stored. In FIG. 2, only components related to embodiments of the present invention are shown. Therefore, those skilled in the technical field to which the present invention pertains should appreciate that general-use components other than the components shown in FIG. 2 may be additionally included.

According to the embodiment of the present invention, the processor 110 controls overall operations of the computing device 100. The processor 110 may provide a user with appropriate information or a function or process the information or function by processing a signal, data, information, etc., input or output through the foregoing components or running an application program stored in the memory 120.

In addition, the processor 110 may perform computation for at least one application or program for executing a method according to embodiments of the present invention, and the computing device 100 may include at least one processor.

According to the embodiment of the present invention, the processor 110 may be composed of one or more cores and may include a processor for data analysis and deep learning (DL) such as a central processing unit (CPU), a general-purpose graphics processing unit (GPGPU), a tensor processing unit (TPU), etc., of a computing device.

The processor 110 may read a computer program stored in the memory 120 and perform data processing for an artificial intelligence (AI) model in accordance with the embodiment of the present invention. According to the embodiment of the present invention, the processor 110 may perform computation to train a neural network. In DL, the processor 110 may perform calculations for training a neural network such as processing input data for training, extracting features from the input data, calculating errors, and updating weights of the neural network using backpropagation.

Also, as the processor 110, at least one of the CPU, the GPGPU, and the TPU may process training of a network function. For example, the CPU and GPGPU may process together training of the network function and data classification based on the network function. Further, according to the embodiment of the present invention, processors of a plurality of computing devices may be used together to process training of the network function and data classification based on the network function. In addition, a computer program executed in a computing device according to the embodiment of the present invention may be a program executable by a CPU, a GPGPU, or a TPU.

In this specification, the term “network function” may be used interchangeably with “artificial neural network” or “neural network.” In this specification, a network function may include one or more neural networks, and in this case, an output of the network function may be an ensemble of outputs of the one or more neural networks.

The processor 110 may read a computer program stored in the memory 120 to provide a classification model in accordance with the embodiment of the present invention. According to the embodiment of the present invention, the processor 110 may perform calculations for training the classification model.

According to the embodiment of the present invention, the processor 110 may generally process overall operations of the computing device 100. The processor 110 may process a signal, data, information, etc., input or output through the foregoing components or run an application program stored in the memory 120, thereby providing appropriate information or a function to a user or a user terminal or processing the information or function.

In addition, the processor 110 may perform computation for at least one application or program for performing methods according to embodiments of the present invention, and the computing device 100 may include one or more processors.

In various embodiments, the processor 110 may further include a random access memory (RAM; not shown) and a read-only memory (ROM; not shown) which temporarily and/or permanently store a signal (or data) processed in the processor 110. Also, the processor 110 may be implemented in the form of a system on chip (SoC) including at least one of a GPU, a RAM, and a ROM.

The memory 120 stores various data, commands, and/or information. The computer program 151 may be loaded from the storage 150 into the memory 120 to perform methods/operations according to various embodiments of the present invention. When the computer program 151 is loaded into the memory 120, the processor 110 may perform the methods/operations by executing one or more instructions constituting the computer program 151. The memory 120 may be implemented as a volatile memory such as a RAM, but the technical scope of the present disclosure is not limited thereto.

The bus 130 provides a communication function between the components of the computing device 100. The bus 130 may be implemented in various forms of buses such as an address bus, a data bus, a control bus, and the like.

The communication interface 140 supports wired or wireless Internet communication of the computing device 100. Also, the communication interface 140 may support various communication methods in addition to Internet communication. To this end, the communication interface 140 may include a communication module well known in the technical field of the present invention. In some embodiments, the communication interface 140 may be omitted.

The storage 150 may non-temporarily store the computer program 151. When the computing device 100 performs a process for performing the multi-model-based FTP estimation method, the storage 150 may store various information required for providing the process for performing the multi-model-based FTP estimation method.

The storage 150 may include a non-volatile memory, such as a ROM, an erasable programmable ROM (EPROM), an electrically erasable programmable ROM (EEPROM), a flash memory, etc., a hard disk, a removable disk, or any form of computer-readable recording medium well known in the technical field to which the present invention pertains.

The computer program 151 may include one or more instructions that, when loaded into the memory 120, cause the processor 110 to perform methods/operations according to various embodiments of the present invention. In other words, the processor 110 may perform the methods/operations according to various embodiments of the present invention by executing the one or more instructions.

In the embodiment, the computer program 151 may include one or more instructions for performing the multi-model-based FTP estimation method including a step of acquiring time-series user data, a step of selecting one model among a plurality of FTP estimation models as a user-customized FTP estimation model on the basis of the time-series user data, and a step of utilizing the user-customized FTP estimation model to generate FTP information.

Steps of the method or algorithm described in connection with an embodiment of the present invention may be directly implemented using hardware, implemented using software modules executed by hardware, or implemented using a combination thereof. The software modules may reside in a RAM, a ROM, an EPROM, an EEPROM, a flash memory, a hard disk, a removable disk, a compact disc (CD)-ROM, or any form of a computer-readable recording medium well known in the technical field to which the present invention pertains.

Components of the present invention may be implemented as a program (or application) and stored in a medium to be executed in combination with a computer which is hardware. Components of the present invention may be executed by software programming or software elements. Similarly, an embodiment may be implemented in a programming or scripting language, such as C, C++, Java, an assembler, etc., to include various algorithms which are embodied as combinations of data structures, processes, routines, or other programming elements. Functional aspects may be implemented using an algorithm executed by one or more processors. The multi-model-based FTP estimation method performed by the computing device 100 will be described below with reference to FIGS. 3 to 8.

FIG. 3 is a flowchart illustrating a multi-model-based FTP estimation method according to an embodiment of the present invention. The order of steps shown in FIG. 3 may vary as necessary, and one or more steps may be omitted or added. In other words, the steps included in FIG. 3 are merely an embodiment of the present invention, and the scope of the present invention is not limited thereto.

According to the embodiment of the present invention, the multi-model-based FTP estimation method may include a step S100 of acquiring time-series user data.

In an embodiment, time-series user data is time-series variation data acquired during a user's ride and may include data based on which the user's exercise intensity or performance at a specific time point or segment during exercise is determined. In the embodiment, the acquired time-series data may be used to evaluate the user's exercise pattern, fatigue level, or exercise capability.

The time-series user data may include time-power data and heart rate data. However, the time-series user data is not limited thereto and may also include data such as travel distance, altitude, location, temperature, air resistance (the coefficient of aerodynamic drag (C_{d}) multiplied by the frontal area (A) (CdA)), speed, pedal revolutions per minute (cadence), and the like. This data may be utilized to evaluate the user's actual riding performance and condition, and may serve as information for various exercise achievement performance analyses and personalized FTP estimation.

According to the embodiment, acquiring the time-series user data may be receiving or loading data stored in the memory 120. Acquiring the time-series user data may be receiving or loading a plurality of pieces of training data from another computing device or a separate processing module in the same computing device to another storage medium on the basis of a wired/wireless communication means.

According to the embodiment, the process of acquiring time-series user data is performed by external devices collecting data in real time and transmitting the collected data to the computing device 100. Here, the external devices may include exercise achievement measurement devices such as a power meter, a heart rate meter, a speedometer, a GPS device, etc., and in real time, these external devices measure time-power data, heart rate, speed, distance, pedal revolutions (cadence), etc., generated during the user's ride. In an embodiment, the power meter measures power (an output) generated during pedaling on a bicycle, collecting data on power exerted by the user in real time while riding. This data represents how many watts of power are being generated per second and may be utilized as a key metric for evaluating the user's exercise intensity. The heart rate meter measures the user's heart rate in real time and is used to assess how much energy the body is expending and how the body responds to varying exercise intensities. This heart rate data is obtained through a wrist-worn heart rate meter or a chest-strap heart rate meter. The speedometer and the GPS device measure the user's travel distance and speed to record exercise achievement, and collect location information and route data in real time during the ride. The collected data is transmitted to the computing device 100 through a wired/wireless communication means, and the computing device 100 analyzes the user's exercise intensity, fatigue level, and achievement on the basis of the received data and utilizes the received data to select an optimal FTP estimation model.

According to the embodiment of the present invention, the multi-model-based FTP estimation method may include a step S200 of selecting one model among a plurality of FTP estimation models as a user-customized FTP estimation model on the basis of the time-series user data.

According to an embodiment, the plurality of FTP estimation models may be intended to generate FTP information more precisely by analyzing the user's riding pattern, heart rate, and power data in various aspects. For example, when only one FTP estimation (or calculation) method is utilized, inaccurate FTP information which is unsuitable for some users may be derived, or various exercise circumstances may not be reflected.

As a specific example, an FTP estimation method employing a single model is suitable for a specific test protocol or riding situation, and thus it is difficult to accurately estimate FTP on the basis of general riding data. Also, when only a heart rate-based estimation method is used, the reliability of FTP information may be degraded because FTP varies depending on environmental changes, each individual's heart rate response pattern, and stored information values such as resting heart rate, maximum heart rate, and the like.

Therefore, the present invention utilizes a plurality of FTP estimation models for estimating a user's FTP in different ways. These models may derive more precise FTP information by processing a user's riding pattern, power output, and heart rate data differently.

In other words, the plurality of FTP estimation models estimate FTP information in different ways by analyzing time-series user data using different methods. Since the models utilize different data and different calculation methods, the best-suited model can be selected in accordance with a user's actual riding data and situation to provide accurate FTP information.

In a specific embodiment, the plurality of FTP estimation models may include a first model for estimating FTP on the basis of time-series data corresponding to an FTP test protocol, a second model for estimating FTP on the basis of MMP data, and a third model for estimating FTP on the basis of the correlation between heart rate data and power data.

According to the embodiment of the present invention, the first model may calculate an average power corresponding to a specific riding interval included in the time-series data, identify a type of test in accordance with the riding pattern, identify a correction factor corresponding to the identified type of test, and generate FTP information on the basis of the calculated average power and the identified correction factor.

According to an embodiment, the first model may be a tested FTP model that, when a user performs a test in accordance with a traditional FTP protocol, estimates time-series power data by utilizing an FTP formula.

Specifically, the first model may generate an average power on the basis of time-power data generated during the user's ride. First, the first model calculates an average power of the specific riding interval from time-series data that is input when the user performs the FTP test. Subsequently, a type of test, such as a 20-minute FTP test or a ramp test, is identified depending on a riding pattern, and the correction factor is applied in accordance with the identified type of test. For example, in the case of a 20-minute FTP test, a corrector factor of 0.95 may be applied to calculate a final FTP value. This process is intended to correct instantaneous power changes that occur during the test, and estimate an FTP value of an exercise intensity threshold at which a lactate level in the user's body remains stable during the user's exercise.

For example, the correction factor of 0.95 may be applied to a 20-minute FTP test on the basis of data of the user riding at maximal effort for 20 minutes, and the correction factor of 0.71 may be applied to a ramp test on the basis of gradually increasing power output.

In an embodiment, the first model may personalize and apply the correction factor on the basis of individual information such as a user's fitness level, training data, age, and gender, and peak power statistics and training status of other users. For example, the correction factor may be finely adjusted in accordance with a specific user's training intensity or fatigue level, providing a more personalized FTP value. As a specific example, when a user is estimated to have higher anaerobic peak power for 15 seconds, 30 seconds, 1 minute, etc., than other users with similar FTP, a shorter TTE may be applied in consideration of the user's anaerobic energy capability instead of a general correction factor, and a higher FTP estimation value may be provided. On the other hand, when a user is estimated to have higher aerobic peak power for 10 minutes, 20 minutes, 30 minutes, etc., than other users with similar FTP, a longer TTE may be applied using the correction factor, and a low FTP estimation value may be provided.

As another example, the correction factor may vary depending on physical differences based on the user's age and gender. For example, for older users, who have relatively lower anaerobic energy, the correction factor may be lowered to extend TTE and provide a lower FTP value, and for younger users, who have greater anaerobic energy, TTE may be shortened to estimate a high FTP value. Considering statistical differences in physical fitness between genders where women generally have lower anaerobic capacity and higher aerobic capacity than men, it is also possible to apply correction factors for extending TTE and reducing FTP to better suit women.

This personalized correction contributes to deriving FTP information that aligns much more closely with a user's actual experience than existing uniform FTP methods.

In other words, the first model analyzes power data generated on the basis of various FTP test protocols in a riding interval and combines the power data with the correction factor to generate final FTP information. This first model may be advantageous for deriving an accurate FTP value in a standardized testing environment.

According to the embodiment of the present invention, the second model may analyze the user's weight and riding interval data on the basis of MMP data to derive correlation information between the MMP and the weight, generate a power duration curve on the basis of the derived correlation information, and generate FTP information on the basis of the generated power duration curve.

In an embodiment, the second model is a statistical model that applies correction statistics to the user's MMP and weight, and may be a weighted FTP model.

Specifically, the second model analyzes maximum power generated during a specific period in the user's riding interval using MMP data. MMP is the highest power that a user can hold for a specific duration, and is utilized as a key element for evaluating the user's riding performance. In an embodiment, MMP refers to the average power of maximal effort that can be exerted by a user during a specific period. For example, when a subject continuously exerts 400 watts for two minutes through maximal effort, the user's 2-minute peak power may be recorded as 400 watts. A criterion for determining maximal effort may be measured during a period in which the user maintains the maximum force that he or she can exert.

The second model generates a power-duration curve on the basis of the correlation between MMP and weight. The second model may detect that no power data falls below a set threshold in relation to existing FTP through a moving average process corresponding to a set time (e.g., 30 seconds) for specific interval time-series data on the basis of the MMP. In the embodiment, the second model may detect that no power data falls below 80% in relation to existing FTP through a 30-second moving average process for corresponding interval time-series power data. When a time during which no data shows a power drop of 80% or less in relation to the existing FTP as described above is maintained for a certain period (e.g., 4 minutes) or longer, this is primarily considered as an intentional action. Subsequently, the user's weight and riding interval data are analyzed to derive information on a relationship between peak power and weight. When w/kg derived for a specific period is higher than w/kg for the same period in the past, a power-duration curve may be generated on the basis of previously stored statistical information.

A power-duration curve is a curve that visualizes a user's power output in accordance with various time intervals, enabling prediction of power reduction based on a riding time. For example, a power-duration curve reflects a pattern where peak power may be very high for a short period but gradually decreases over time. A power-duration curve enables a user to predict power that may be exerted in each riding time interval, and accurate FTP information can be derived from the predicted power.

For example, the second model generates a power-duration curve on the basis of a period in which the MMP is recorded and the weight data to predict power that may be exerted by the user in various time intervals, and calculates FTP from the predicted power. As an example, a suitable time for the subject is identified within a period between 20 minutes and 80 minutes on the basis of the generated power-duration curve to calculate estimated power, and FTP information may be generated on the basis of the estimated power. MMP occurs during a short burst of high-intensity riding, allowing the estimation of each individual's FTP value that may be sustained for his or her TTE.

Also, the second model may adjust an FTP value in accordance with weight change using the power-duration curve. For example, when the user's weight increases at peak power of a specific period, FTP may be calculated by considering a reduction in aerobic power, and when the weight decreases, higher FTP may be calculated by assuming an increase in aerobic power. In this way, the user's weight value relative to the same peak power may be utilized as a value for adjusting a slope of the power duration curve by adjusting an aerobic-to-anaerobic ratio.

As another example, users in their twenties may generate higher peak power during a short time interval compared to those in their fifties. Therefore, a power-duration curve for them reflects high peak power output during a short time interval of 10 minutes or less. On the other hand, a power-duration curve for older users reflects lower output during a short time interval of 10 minutes or less even at the same peak power/weight. By applying these age-specific statistics for fine-tuning, it is possible to achieve a more realistic FTP estimation. This also applies to gender, resulting in lower peak power estimates for women than men during a short time interval.

In other words, the second model is a model that generates a power-duration curve on the basis of the correlation between MMP data and weight and provides FTP information accurately reflecting the user's power changes in accordance with time intervals from the power-duration curve.

According to the embodiment of the present invention, the third model calculates an average power of a stable heart-rate interval by analyzing a correlation between the user's heart rate and power and estimates FTP information on the basis of the calculated average power and the user's maximum heart rate.

In an embodiment, the third model may be a submaximal FTP model that detects a time-series data segment in which riding is performed at a stable heart rate on the basis of the linear relationship between heart rate and power and estimates FTP using an average power of the segment. By analyzing power data measured within a stable heart rate range, normalized FTP information is derived on the basis of power generated when the user is within the 70% to 90% range of his or her maximum heart rate.

According to the embodiment, the third model detects an interval in which the user rides at a stable heart rate by analyzing the linear relationship between heart rate (HR) and power on the basis of a submaximal FTP estimation model of Alan Couzens and estimates FTP information on the basis of an average power data of the interval. An existing submaximal FTP model calculates an average power value of an activity interval in which a stable heart rate of 70% or more of the maximum heart rate is maintained, and estimates about 87% of the maximum heart rate as FTP.

However, the existing submaximal FTP model has an issue in that FTP values are overestimated or underestimated due to delayed or inaccurately measured heart rates which are caused by significant individual differences in resting and maximum heart rates based on activity levels, unstable wearing of the heart rate meter, and environmental factors such as temperature, a user's condition, or the like. To address this, the third model of the present invention defines the resting heart rate as 55% of the maximum heart rate when the resting heart rate is unclear. The maximum heart rate is defined as a maximum heart rate directly set by the user as the top priority, but when no user-defined maximum heart rate exists, 180 is defined as a reference value. When a stable heart rate value in the riding data is less than 70% of the maximum heart rate, the stable heart rate value is not utilized in FTP estimation.

In an embodiment, the third model may perform a moving average process corresponding to a time (30 seconds or 1 minute) set for the user's heart rate and power and detect an interval of data subjected to the moving average process in which a heart rate is not 70% or more of the maximum heart rate and power does not fall below 80% or less in relation to the existing FTP. The third model may analyze the correlation between power and heart rate of an identified interval to calculate the maximum power matching the existing user's maximum heart rate and may multiply the calculated maximum power by a coefficient of 0.86 to 0.88 to generate FTP information.

In an embodiment, the third model normalizes power data using a 1-minute moving average to correct for temporary fluctuations, removes device errors and environmental factors, and then derives an FTP value. Through this preprocessing process, more reliable data is ensured, and it is possible to estimate accurate FTP by utilizing peak power for a specific time and corresponding heart rate data.

For example, generated power data may be analyzed on the basis of data in which a heart rate is stably maintained within a 70% to 90% range of the maximum heart rate, and an FTP value may be estimated on the basis of an average power within that range. By applying a 1-minute moving average of power data, it is possible to minimize instability of data caused by heart rate changes and estimate accurate FTP.

In addition, to correct measurement errors that may occur due to unstable wearing of the heart rate meter, an FTP value is calculated by utilizing the normalized power data only for a data segment in which an effort level is moderate or higher, corresponding to 70% or more of the maximum heart rate. Accordingly, it is possible to prevent the overestimation issue of the existing submaximal FTP model.

Therefore, the third model analyzes the correlation between heart rate and power to provide personalized FTP information on the basis of an average power of an interval where a heart rate is stably maintained at a moderate intensity or higher, and enables realistic and reliable FTP estimation for the user irrespective of success or failure in a high-intensity test and an FTP test.

As described above, the computing device 100 of the present invention can provide a user with customized FTP information by utilizing a plurality of FTP estimation models. For example, when the user performs a high-intensity FTP test, the first model is selected, and when the user records MMP, the second model is selected. On the other hand, when it is necessary to estimate FTP from the correlation between heart rate data and power in a general riding situation without performing a maximal intensity test, the third model is selected.

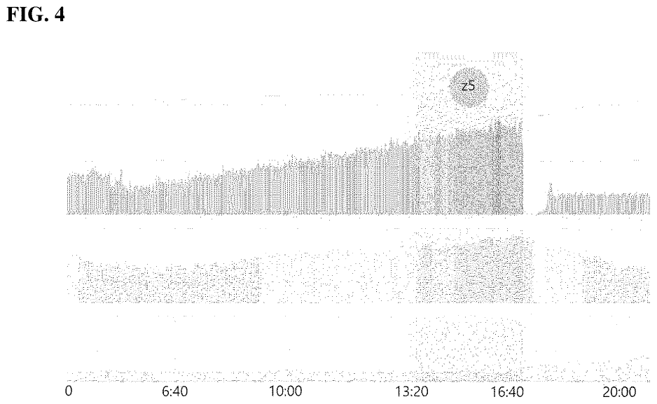

As a specific example, FIG. 4 shows a high-intensity FTP test case. The X axis represents riding time, the Y axis represents power output (watts) and heart rate (bpm), and high-intensity and low-intensity intervals are distinguished by different colors. FIG. 4 visually shows changes of power data and heart rate data generated during a ride. For example, in the high-intensity interval, it is possible to see a pattern where power output rapidly increases and heart rate also increases accordingly. In this interval, the first model is applied to estimate the user's FTP information.

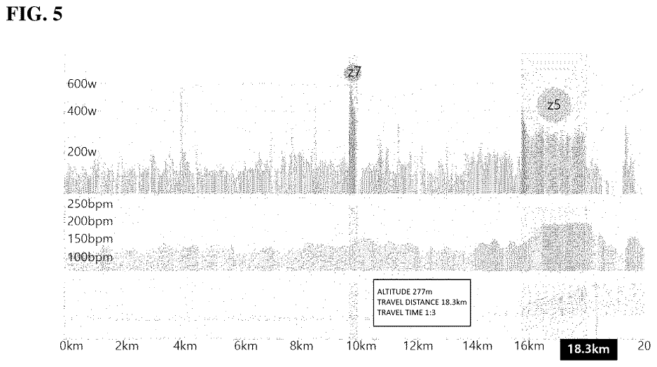

Also, FIG. 5 shows intermittent interval riding, which may be a suitable case for the third model. The X axis represents travel distance (km), and the Y axis represents power output (watts) and heart rate (bpm). FIG. 5 visualizes a case where a user shows an irregular riding pattern and there is no certain correlation between heart rate and power. In this case, no fixed test protocol is applied, and thus the third model may be selected to derive FTP information by analyzing the correlation between heart rate and power.

In this way, the first model and the second model are utilized when a test protocol is clearly performed or high-intensity peak power data is recorded. For example, when a user performs a 20-minute FTP test or records maximum peak power, FTP information is derived through the first model or the second model. On the other hand, the third model analyzes data generated from daily rides in real time and dynamically calculates an FTP value on the basis of a riding pattern and heart rate changes. This enables the provision of a reliable FTP value based on various data collected from a ride even when a user does not obviously perform a test.

The computing device 100 statistically analyzes time-series time-power data accumulated by the user to determine whether actual peak power is intentionally exerted with maximal effort or whether there is an FTP test, and then dynamically selects the best-suited FTP estimation model depending on the determination result. Through this process, it is possible to provide accurate FTP information aligned with the user's individual.

In other words, the computing device 100 can determine whether the user actually exerts effort by selecting a suitable FTP estimation model for each situation on the basis of the user's riding data and heart rate data and generate optimized FTP information accordingly. Therefore, not only professional athletes but also general users can easily obtain suitable FTP information, and a reliable FTP value is provided from daily riding data without a high-intensity test.

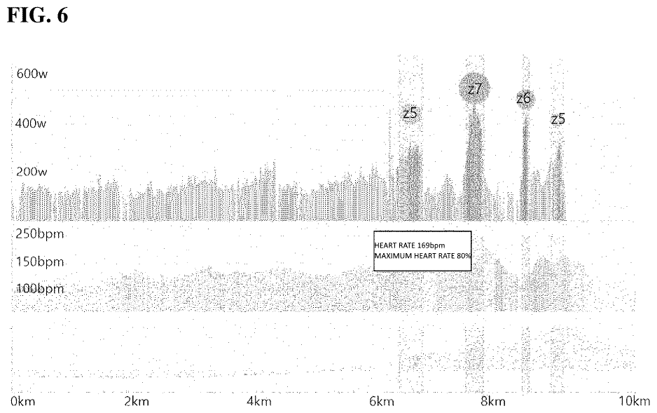

For this reason, it is possible to provide accurate and customized FTP estimation to various users by minimizing data bias without depending on an existing fixed test protocol. Further, in an embodiment, the computing device 100 can utilize a classification model to analyze the user's riding pattern and heart rate data and detect whether maximal effort is intentionally exerted. Referring to FIG. 6, a user's specific riding intervals show that the heart rate and power output rapidly increase. In FIG. 6, the X axis represents travel distance (km) and the Y axis represents power output (watts) and heart rate (bpm). FIG. 6 shows rapid increases in heart rate and power output at high-intensity intervals. For example, in an interval in which the heart rate reaches 169 bpm that is 80% of the maximum heart rate, the classification model may detect that the user intentionally continues high-intensity effort.

When intentional effort is detected, the classification model selects the first model or the second model, estimating FTP information on the basis of the fact that the user performs a high-intensity FTP test or records MMP. On the other hand, in FIG. 6, intervals in which the relation between heart rate and power is not kept relatively uniform may be detected as unintended riding patterns. In this case, the third model may be selected to dynamically estimate an FTP value on the basis of heart rate and power data.

Consequently, the computing device selects a suitable model for each riding situation through the classification model for determining whether intentional effort is exerted, providing optimal FTP information. In this way, the user can estimate an accurate FTP value from daily riding data without depending on a high-intensity test protocol.

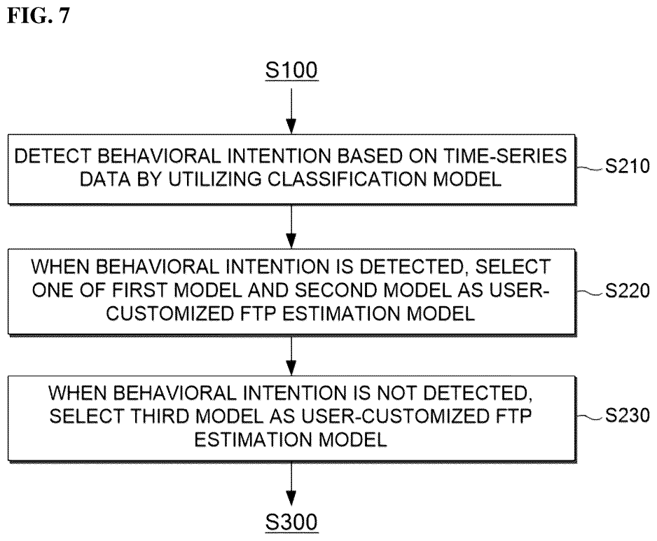

In a specific embodiment, referring to FIG. 7, the step of selecting one model among the plurality of FTP estimation models may include a step S210 of detecting behavioral intention on the basis of the time-series user data by utilizing the classification model, a step S220 of selecting one of the first model and the second model as the user-customized FTP estimation model when behavioral intention is detected, and a step S230 of selecting the third model as the user-customized FTP estimation model when behavioral intention is not detected.

Here, the behavioral intention may include information related to whether maximal effort is intentionally exerted and whether an FTP test is performed.

In the embodiment, the classification model is a neural network model that is pretrained using a training dataset including a plurality of users' time-series data, riding data, and intention information corresponding thereto, and analyzes the time-series user data to detect the behavioral intention.

In an embodiment, the classification model is a machine learning-based neural network model and may be pretrained using a training dataset including a plurality of users' time-series data, riding data, and intention information. This classification model serves to detect whether maximal effort is intentionally exerted or whether an FTP test is performed by analyzing time-series user data and heart rate data. During a training process, the classification model develops its capability to detect behavioral intention on the basis of various users' riding patterns and data.

For example, when an intentional FTP test is performed, a stepwise increase in power output of exactly 20 watts per minute or a steady high-power output maintained for approximately 20 minutes may be detected in time-series data, and when MMP is recorded, a rapid increase in power lasting over 4 minutes and corresponding changes in heart rate occur. These patterns are repeatedly analyzed during the model training process and used as criteria for the classification model to detect behavioral intention.

Also, in an embodiment, the classification model analyzes the correlation between the riding pattern and the heart rate pattern on the basis of the pretrained neural network and accordingly determines whether the user intentionally exerts maximal effort. For example, when the user's time-series data shows a pattern in which the power output rapidly increases and the heart rate increases together in a specific interval, it is detected that an FTP test is performed. On the other hand, when power output changes are inconsistent and heart rate variations are minimal, the classification model may recognize this case as a daily ride and select the third model.

As a result, the classification model selects an optimal model for user-customized FTP estimation to provide accurate FTP information suitable for each situation.

More specifically, in step S210, the computing device 100 detects the user's behavioral intention by analyzing the time-series user data and heart rate data. The classification model analyzes the correlation between the riding pattern and the heart rate pattern on the basis of the pretrained neural network, thereby determining whether the user intentionally exerts maximal effort or whether the user performs an FTP test.

For example, when the user's time-series data shows a pattern for approximately 20 minutes in which the power output is kept high and the heart rate is also kept high in a specific interval, it is detected that an FTP test is performed. On the other hand, when power output changes are inconsistent and heart rate variations are minimal, this is recognized as a daily ride, and the third model is selected.

In operation S220, when behavioral intention is detected, the computing device 100 may select one of the first model and the second model as a user-customized FTP estimation model.

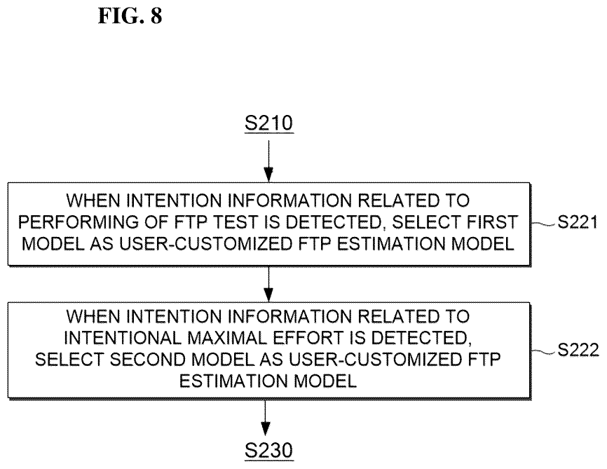

More specifically, referring to FIG. 8, when behavioral intention is detected, the step S220 of selecting one of the first model and the second model as a user-customized FTP estimation model may include a step S221 of selecting the first model as the user-customized FTP estimation model when intention information related to performing of an FTP test is detected, and a step S222 of selecting the second model as the user-customized FTP estimation model when intention information related to intentional maximal effort is detected.

More specifically, when intention information related to performing of an FTP test is detected, the first model is selected. Here, the first model estimates FTP information by applying a correction factor corresponding to a specific test protocol on the basis of data of a standard FTP test performed by the user. For example, in the base of a 20-minute FTP test, a correction factor of 0.95 may be applied to calculate an FTP value.

Meanwhile, when intention information related to intentional maximal effort is detected, the second model is selected. The second model generates a power-duration curve on the basis of the MMP data and the weight and estimates FTP information by utilizing the power-duration curve. This approach is based on maximal power data of a short time and provides a more suitable FTP value in consideration of the user's various physical conditions.

Meanwhile, when no behavioral intention is detected in operation S230, the computing device 100 may select the third model as a user-customized estimation model. The third model corresponds to a method of analyzing the correlation between heart rate and power to estimate FTP information on the basis of a user's daily riding data. This model calculates an FTP value on the basis of power data generated from the 70% to 90% range of the maximum heart rate, and in this way, it is possible to obtain accurate FTP information in a daily riding environment.

As described above, the computing device 100 comprehensively analyzes a user's real-time riding pattern and heart rate data by utilizing a classification model and dynamically selects an optimal FTP estimation model. In this way, it is possible to provide optimal FTP information suitable for each situation and estimate personalized FTP. When the user's intentional action is detected, a suitable model may be selected to derive accurate FTP information, leading to a reduction in dependency on a fixed test protocol. As a result, the method has an advantage of operating in a suitable way not only for professional athletes but also for general users, and it is possible to provide a flexible FTP estimation method that is suitable for users of all levels.

According to the embodiment of the present invention, the multi-model-based FTP estimation method may include a step S300 of generating FTP information by utilizing the user-customized FTP estimation model. The computing device 100 processes the user's time-series data and heart rate data on the basis of the FTP estimation model selected by utilizing the classification model (i.e., the user-customized FTP estimation model), thereby calculating optimal FTP information. The computing device 100 provides personalized FTP information reflecting each user's riding pattern and physical condition and thus can calculate and provide an accurate and reliable FTP value in not only daily rides but also intentional test situations.

According to the embodiment, outputs of the plurality of models may have priorities in an order of the first model, the second model, and the third model. This priority order is set to provide the most reliable result to the user in accordance with the accuracy and applicable situation of each model.

This configuration is required for more accurately reflecting high-intensity data generated when the user intentionally exerts maximal effort or in a test situation. The first model can estimate the most reliable FTP on the basis of an intentionally executed FTP test protocol and thus is primarily selected. The second model reflects maximal effort recorded for a short period on the basis of the user's MMP, thus efficiently estimating FTP even from data of a short period compared to a test situation. On the other hand, the third model is suitable for providing stable FTP information in general situations by utilizing the correlation between heart rate and power that appears during daily rides.

The reason for prioritization may be that the models are optimized on the basis of data collected in different situations. For example, when the user performs a 20-minute FTP test, the first model can provide the most accurate result, but when the user records MMP for a short period or in a field, the second model is more suitable. The third model advantageously operates for daily training or in a situation requiring heart rate-based estimation.

The advantage of this prioritization is that it is possible to provide optimal FTP information suitable for the user's situation. In this way, the user can automatically obtain suitable FTP information even during daily training and can be provided with accurate results after performing an intentional FTP test. Also, even when the user exerts maximal effort over 4 minutes for a short period, analysis results can be rapidly obtained through the second model.

As a result, the computing device 100 can provide optimal FTP estimation results across various riding situations by utilizing advantages and characteristics of each model on the basis of the priority order. This enables the implementation of a personalized FTP estimation system suitable not only for professional athletes but also for general users.

In addition, the computing device 100 can dynamically update FTP information on the basis of data collected in real time and provide personalized feedback in consideration of the user's training intensity and fatigue level, contributing to designing an efficient exercise plan.

According to the embodiment, the multi-model-based FTP estimation method may include a step of updating the FTP information only when higher FTP information than FTP information of a previous time point is generated. This configuration allows an update only when the user's performance improvement is clearly proved, preventing FTP from being overestimated due to inaccurate data.

More specifically, the computing device 100 analyzes time-series data collected in real time during the user's ride and updates the FTP information when the user intentionally exerts effort or training performance is improved. For example, when the user records new peak power or accumulates long-term training results and exceeds his or her existing FTP, a previous FTP value is updated with new FTP information. On the other hand, when performance declines due to fatigue during training or a temporary data error occurs, existing FTP information is maintained.

This update method is designed to accurately reflect changes in the user's fitness while avoiding excessive reflection of a performance decline caused by temporary deconditioning. This enables stable performance management and provides a system that supports continuous performance improvement over a long term.

Also, in an embodiment, the multi-model-based FTP estimation method may further include a step of estimating TTE information on the basis of the FTP information and the weight data and a step of correcting the TTE information by applying an anaerobic weight on the basis of user information to generate customized TTE information. Here, the customized TTE information may be information defining a time for which the user can maintain maximal power.

More specifically, the computing device 100 may calculate TTE information on the basis of the user's FTP information and weight data. The TTE information is defined as a time for which the user can maintain FTP. In general, the TTE information of athletes is close to 1 hour, but that of common people or less trained people may widely vary. Therefore, it is important to accurately estimate FTP and TTE that is a time for which the FTP is maintained. An embodiment of the present invention provides not only existing FTP information but also user-customized TTE information, enabling an improvement in training performance.

During a processing of estimating TTE, the computing device 100 may utilize a formula for calculating a TTE on the basis of a user weight and an FTP value. For example, a TTE may be calculated as TTE=(W/kg for 1 hour)×10+5. Through this calculation, the user is provided with information about how long he or she can continue exercising at a specific intensity.

Also, an anaerobic weight based on the user information such as age and gender is applied to the TTE such that an accurate maintenance time based on the individual's physical characteristics is derived. For example, using an age of 40 as a reference, a linear calculation formula may be applied such that a TTE of a 25-year-old user is reduced by 10% while a TTE of a 55-year-old user is increased by 10%. Depending on gender, a weight may be applied to increase TTEs of women by approximately 10%. These weights enable the provision of customized TTE information reflecting physical differences based on age and gender.

As a specific example, when an FTP of a 30-year-old male weighing 65 kg is 260 watts, a TTE may be calculated using the following formula.

TTE=((260/65)×10±5)×0.93333

In this way, the TTE is derived as about 42 minutes. In other words, the user can maintain his FTP for about 42 minutes.

As a result, the computing device 100 calculates personalized TTE information on the basis of the user's weight, age, gender, and FTP value, which may play an important role in establishing a training plan and managing performance.

In summary, the computing device 100 may comprehensively analyze a user's weight, age, gender, FTP value, and existing riding data and provide optimized TTE information which is customized for each user. In this way, the user can design a training plan suited for his or her physical characteristics and efficiently manage performance on the basis of accurate information about how long he or she can maintain given power. Also, the computing device 100 collects and analyzes data in real time and dynamically updates TTE in accordance with changes in the user's physical condition and training intensity, thereby supporting the setting and achievement of a customized training goal.

The present invention enables a systematic power-based aerobic exercise management system to be utilized more universally by naturally estimating and providing functional threshold power (FTP) during a daily cycling exercise, eliminating the need to follow a fixed test protocol. Accordingly, not only professional athletes but also general users can design efficient training plans and manage performance on the basis of FTP and time to exhaustion (TTE).

Further, according to the present invention, it is possible overcome a painful testing protocol and data bias issues centered on trained athletes that are required in existing FTP estimation methods. This enables a broader range of general users to utilize an FTP-based aerobic exercise management model, which is only used by a small number of elite athletes or advanced cycling enthusiasts.

In addition, the present invention estimates not only FTP information but also TTE information, enabling estimation of a time for which a user can maintain FTP. This may be utilized as a variable closely correlated with the user's resilience, contributing to more efficient performance management by utilizing performance and even a resilience variable during an extended ride

Effects of the present invention are not limited to those described above, and other effects that have not been described will be clearly understood by those of ordinary skill in the art from the following description.

Operations of a method or algorithm described in connection with an embodiment of the present invention may be directly implemented as hardware, implemented as a software module executed by hardware, or implemented as a combination of hardware and a software module. The software module may be on a RAM, a ROM, an EPROM, an EEPROM, a flash memory, a hard disk, a removable disk, a compact disc (CD)-ROM, or any type of computer-readable recording medium well known in the technical field to which the present invention pertains.

Components of the present invention may be implemented as a program (or an application) and stored in a medium to be executed in combination with a computer which is hardware. Components of the present invention may be implemented by software programming or software modules. Similarly, embodiments may be implemented in a programming or scripting language, such as C, C++, Java, assembly language, or the like, to include various algorithms implemented as combinations of data structures, processes, routines, or combinations of other programming elements. Functional aspects may be implemented using an algorithm that is executed by one or more processors.

Those skilled in the art of the present invention should understand that various exemplary logic blocks, modules, processors, means, circuits, and algorithm steps described in connection with the embodiments described herein may be implemented by electronic hardware, various forms of programs or design codes (for convenience, referred to as “software” herein), or a combination thereof. To clearly show the compatibility of hardware and software, a variety of illustrative components, blocks, modules, circuits, and steps have been generally described above in connection with functions thereof. Whether these functions are implemented by hardware or software is related to a particular application and design constraints of the whole system. Those skilled in the art can implement a function described in a variety of ways for each specific application, but the decision of the implementation should not be construed as departing from the scope of the present invention.

The variety of embodiments set forth herein may be implemented as articles manufactured using methods, devices, or standard programming and/or engineering techniques. The term “manufactured article” includes a computer program, a carrier, or a medium accessible by any computer-readable device. Examples of a computer-readable medium may include, but are not limited to, magnetic storage devices (e.g., a hard disk, a floppy disk, a magnetic strip, etc.), optical discs (e.g., a CD, a digital versatile disc (DVD), etc.), smart cards, and flash memory devices (e.g., an EEPROM, a card, a stick, a key drive, etc.). In addition, the variety of storage media presented herein include one or more devices for storing information and/or other machine-readable media. The term “machine-readable media” includes, but is not limited to, wireless channels and various other media for storing, retaining, and/or transmitting an instruction and/or data.

It should be understood that the specific order or hierarchical structure of steps of each of the presented processes is an example of illustrative approaches. It should be understood that a specific order or hierarchical structure of steps of processes within the scope of the present disclosure may be rearranged on the basis of design priorities. The appended method claims provide elements of various steps in a sample order but are not limited to the specific order or hierarchical structure presented herein.

Description of the embodiments set forth herein is provided to help those of ordinary skill in the art use or implement the present invention. It will be obvious to those of ordinary skill in the technical field of the present invention that various modifications can be made from these embodiments, and the general principles defined herein may be applied to other embodiments without departing from the scope of the present invention. Therefore, the present invention is not limited to the embodiments set forth herein but should be construed in the broadest scope consistent with the principles and novel features presented herein.

What is claimed is:

1. A method of estimating functional threshold power (FTP) on the basis of multiple models by one or more processors of a computing device, the method comprising:
acquiring time-series user data;
selecting a single model among a plurality of FTP estimation models as a user-customized FTP estimation model on the basis of the time-series user data; and
generating FTP information by utilizing the user-customized FTP estimation model,
wherein the time-series user data includes time-power data and heart rate data generated during a user's ride, and
the plurality of FTP estimation models are models configured to estimate the FTP information in different ways.

2. The method of claim 1, wherein the plurality of FTP estimation models include:
a first model configured to estimate FTP on the basis of time-series data corresponding to an FTP test protocol;
a second model configured to estimate FTP on the basis of mean maximal power (MMP) data; and
a third model configured to estimate FTP on the basis of a correlation between heart rate data and power data.

3. The method of claim 2, wherein the selecting of the single model among the plurality of FTP estimation models comprises:
detecting behavioral intention on the basis of the time-series user data by utilizing a classification model;
when the behavioral intention is detected, selecting one of the first model and the second model as the user-customized FTP estimation model; and
when the behavioral intention is not detected, selecting the third model as the user-customized FTP estimation model,
wherein the behavioral intention includes information related to whether intentional maximal effort is exerted and whether an FTP test is performed.

4. The method of claim 3, wherein the classification model is a neural network model that is pretrained using a training dataset including time-series data and riding data of a plurality of users and intention information corresponding thereto, and detects the behavioral intention by analyzing the time-series user data and the heart rate data.

5. The method of claim 3, wherein the selecting of one of the first model and the second model as the user-customized FTP estimation model when the behavioral intention is detected, comprises:
selecting the first model as the user-customized FTP estimation model when intention information related to performing of the FTP test is detected; and
selecting the second model as the user-customized FTP estimation model when intention information related to the intentional maximal effort is detected.

6. The method of claim 2, wherein the first model calculates an average power corresponding to a specific riding interval included in the time-series data, identifies a type of test in accordance with a riding pattern, identifies a correction factor corresponding to the identified type of test, and generates the FTP information on the basis of the calculated average power and the identified correction factor.

7. The method of claim 2, wherein the second model analyzes the user's weight and riding interval data on the basis of the MMP data to derive correlation information between the MMP and the weight, generates a power duration curve on the basis of the derived correlation information, and generates the FTP information on the basis of the generated power duration curve.

8. The method of claim 2, wherein the third model calculates an average power of a stable heart-rate interval by analyzing a correlation between the user's heart rate and power and estimates FTP information on the basis of the calculated average power and the user's maximum heart rate.

9. The method of claim 2, further comprising updating the FTP information when FTP information higher than FTP information of a previous time point is generated,
wherein outputs of the plurality of models have priorities in an order of the first model, the second model, and the third model.

10. The method of claim 1, further comprising:
estimating time to exhaustion (TTE) information on the basis of the FTP information and weight data; and
applying an anaerobic weight on the basis of user information to correct the TTE information and generate customized TTE information,
wherein the customized TTE information is information defining a time for which a maximum power is maintained by the user.

11. A device, comprising:
a memory configured to store one or more instructions; and
a processor configured to execute the one or more instructions stored in the memory,
wherein the processor performs the method of claim 1 by executing the one or more instructions.

12. A computer-readable recording medium on which a computer program is recorded, wherein the computer program is coupled to a computer which is hardware to execute the method of claim 1.
